(19) United States
(12) Patent Application Publication
(10) Pub. No.: US 2023/0027740 A1
(43) Pub. Date: Jan. 26, 2023

(54) TRIFUNCTIONAL PROCESSES IN CATALYTIC DISTILLATION

(71) Applicant: LUMMUS TECHNOLOGY LLC, Houston, TX (US)

(72) Inventors: Rosette Barias, Houston, TX (US); Liang Chen, Houston, TX (US)

(73) Assignee: LUMMUS TECHNOLOGY LLC, Houston, TX (US)

(21) Appl. No.: 17/813,614

(22) Filed: Jul. 20, 2022

Related U.S. Application Data
(60) Provisional application No. 63/223,614, filed on Jul. 20, 2021.

(51) Int. Cl. C07C 41/09; B01J 8/04; C07C 41/42; B01D 3/00; B01D 3/14

(52) U.S. Cl. CPC C07C 41/09; B01J 8/0492; B01J 8/0446; C07C 41/42; B01D 3/009; B01D 3/143; B01J 2208/00893

(57) ABSTRACT

A process for the production of alkyl ethers including feeding a hydrocarbon feedstock and a first alcohol feedstock to a fixed bed reactor containing an etherification catalyst. The hydrocarbon feedstock and the first alcohol feedstock are contacted in the first fixed bed reactor to react the isoolefins with the alcohol in the presence of the etherification catalyst to produce a first product stream. The first product stream is fed together with a hydrogen feedstock and a second alcohol feedstock to a catalytic distillation reaction system containing a trifunctional catalyst to concurrently isomerize at least a portion of the alpha-olefins, hydrogenate at least a portion of the diolefins, and etherify at least a portion of the isoolefins and alcohol, producing a bottoms product comprising the one or more ethers and an overhead product comprising n-alkanes, isoalkanes, unreacted alpha-olefins, unreacted internal-olefins, unreacted isoolefins, and unreacted alcohol.

### FIELD OF THE DISCLOSURE

Embodiments disclosed herein relate to a catalytic distillation process for producing tertiary alkyl ethers.

### BACKGROUND

The production of tertiary alkyl ethers by the reaction of primary alcohol and an isoolefin is well known in the art. The use of a distillation column reactor to simultaneously react and distill the product from the reactants has been found to be especially beneficial in this normally equilibrium limited reaction. The description of the process using the distillation column reactor and variations thereon are disclosed in commonly assigned U.S. Pat. Nos. 4,218,011; 4,232,177; 4,305,254; 4,504,687; 4,978,807; 5,118,873; 5,120,403; 5,248,836; 5,248,837; and 5,313,005. Catalytic distillation has been widely applied to etherification of isoolefins, which is described in several of the noted patents.

U.S. Pat. No. 5,431,888 discloses a multi-purpose distillation column reactor wherein a hydrogenation catalyst for hydrotreating an isoolefin containing light naphtha from a fluid catalytic cracking unit to remove diolefins and mercaptans is stacked below an etherification catalyst.

Normally the olefin feed to an etherification process is a mixed C₄ stream containing normal and isobutanes, normal and isobutenes, and some butadiene. The isobutene (iC₄⁼) preferentially reacts with a primary alcohol to form one or more ethers such as methyl tert-butyl ether (MTBE) or ethyl tert-butyl ether (ETBE). In a process using C₅s, tert-amyl ethyl ether (TAEE), and/or tert-amyl methyl ether (TAME) may be produced. The unreacted C₄s and C₅s are frequently used as feed stock to a cold acid alkylation process which reacts the normal olefins (e.g., butenes) with isoalkanes (e.g., isobutane) to form alkylate (e.g., isooctane).

Accordingly, processes for etherification of a C₄s using a catalytic distillation column often involves two primary fixed bed reactors. As seen in FIG. 1, a C₄ feed, or other light cut naphtha feed, 2 is combined with fresh or recycle alcohol 4, and hydrogen 6, forming a combined feed 10. The combined feed 10 is fed to a first fixed bed reactor 12 containing one or more beds of one or more catalysts, which together may be capable of etherification of isobutene, positional isomerization of 1-butene to form 2-butene, and hydrogenation of butadiene to form additional n-butenes and n-butanes, forming a first intermediate product having one or more C₄s, one or more ethers, and unreacted alcohol.

The first intermediate product is split into recycle 8 and a feed line 14 to the second fixed bed reactor 16 containing a second catalyst. The second catalyst is a single function catalyst for the etherification of the isobutene and alcohols. The second intermediate product stream 18 may include one or more ethers, unreacted C₄s, and unreacted alcohol.

The second intermediate product stream 18 is then fed to a catalytic distillation reaction system 22 containing a single function etherification catalyst, which may be the same or different than the catalyst in the second fixed bed reactor 16. The catalyst in the catalytic distillation reaction system 22 may also be a combination of the catalyst from the first fixed bed reactor 12 and the second fixed bed reactor 16. Additional alcohol may be fed to the catalytic distillation reaction system 22 via feed line 20 as required for etherification of remaining C₄s in the second intermediate product stream 18.

The catalytic distillation reaction system 22 will react the isobutene and the alcohol to form additional ethers, and separate the one or more ethers from any unreacted C₄s, and unreacted alcohol. The one or more ethers may be recovered via product stream 26 and the unreacted C₄s, excess hydrogen, other lighter hydrocarbons and unreacted alcohol may be recovered via overhead stream 24. Such processes use multiple catalyst beds with multiple types of catalyst in the catalytic distillation reaction system, which are tailored based on the feedstock and desired conversion.

The above-mentioned process is also generally capable of hydrogenation of dienes and isomerization and etherification of C₅ olefins to produce TAME and/or TAEE.

This process requires the first fixed bed reactor 12 to be operated at a relatively high pressure, such as above 15 barg, in order to the keep the combined feed 10 in liquid phase. This is necessary for the noble metal multi-functional catalyst to operate with the desired degree of efficiency. Additionally, this process requires the feed to the first fixed bed reactor 16 to have less than about 2 wt % diolefins in order to avoid poisoning of the etherification catalyst. Accordingly, the feed to the first fixed bed reactor must be relatively low in diolefins, or the hydrogenation reaction within the first fixed bed reactor must be fairly complete.

### SUMMARY OF THE CLAIMED EMBODIMENTS

The present inventors have found that dienes have low reactivity over various etherification catalysts that may be used in the second fixed bed reactor. Accordingly, the processes to convert isoolefins to ethers may be greatly simplified.

In one aspect, embodiments disclosed herein relate to a process for the production of alkyl ethers. The process including feeding a hydrocarbon feedstock comprising n-alkanes, isoalkanes, alpha-olefins, internal-olefins, isoolefins, and diolefins and a first alcohol feedstock to a fixed bed reactor containing an etherification catalyst. The hydrocarbon feedstock and the first alcohol feedstock are contacted in the first fixed bed reactor to react the isoolefins with the alcohol in the presence of the etherification catalyst to produce a first product stream comprising n-alkanes, isoalkanes, alpha-olefins, internal-olefins, unreacted isoolefins, diolefins, and unreacted alcohol and one or more ethers. The first product stream is fed together with a hydrogen feedstock and a second alcohol feedstock to a catalytic distillation reaction system containing a trifunctional catalyst to concurrently isomerize at least a portion of the alpha-olefins, forming additional internal-olefins, hydrogenate at least a portion of the diolefins, forming additional internal-olefins, and etherify at least a portion of the isoolefins and alcohol, forming one or more ethers, producing a bottoms product comprising the one or more ethers and an overhead product comprising n-alkanes, isoalkanes, unreacted alpha-olefins, unreacted internal-olefins, unreacted isoolefins, and unreacted alcohol.

In another aspect, embodiments disclosed herein relate to a system for the production of alkyl ethers. The system including a fixed bed reactor containing an etherification catalyst, where the fixed bed reactor configured for receiving a hydrocarbon feedstock comprising n-alkanes, isoalkanes, alpha-olefins, internal-olefins, isoolefins, and diolefins and a first alcohol feedstock, and contacting the hydrocarbon feedstock and the first alcohol feedstock to produce a first product stream comprising n-alkanes, isoalkanes, alpha-olefins, internal-olefins, unreacted isoolefins, diolefins, and unreacted alcohol and one or more ethers. The system further including a catalytic distillation reaction system containing a single bed of a trifunctional catalyst. The catalytic distillation reaction system further including a first product stream inlet located beneath the single bed of trifunctional catalyst, a hydrogen feedstock inlet located proximate a top of the single bed of trifunctional catalyst, a second alcohol feedstock inlet located proximate a bottom of the single bed of trifunctional catalyst, a bottoms product outlet configured for producing a bottoms product comprising the one or more ethers, and an overhead product outlet configured for producing an overhead product comprising n-alkanes, isoalkanes, unreacted alpha-olefins, unreacted internal-olefins, unreacted isoolefins, and unreacted alcohol. The catalytic distillation reaction system is configured to concurrently isomerize at least a portion of the alpha-olefins, forming additional internal-olefins, hydrogenate at least a portion of the diolefins, forming additional internal-olefins, and etherify at least a portion of the isoolefins and alcohol, forming one or more ethers.

Other aspects and advantages will be apparent from the following description and the appended claims.

### BRIEF DESCRIPTION OF DRAWINGS

FIG. 1 is a process flow diagram which illustrates a prior art process for etherification.

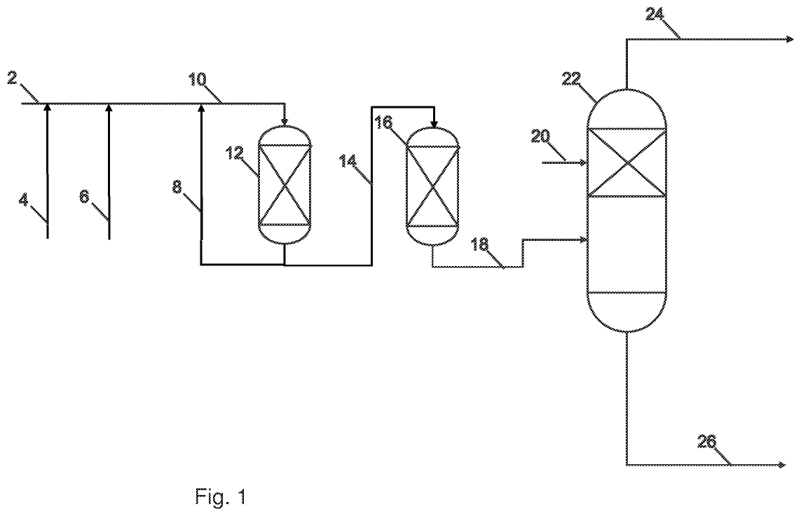

FIG. 2 is a process flow diagram which illustrates an etherification process according to one or more embodiments disclosed herein.

### DETAILED DESCRIPTION

Embodiments herein relate generally to systems and processes for etherification of isoolefins.

As used in embodiments disclosed herein, “catalytic distillation reaction system” or like terms refers to a system for concurrently reacting compounds and separating the reactants and the products using fractional distillation. In some embodiments, the catalytic distillation reaction system may comprise a conventional catalytic distillation column reactor, where the reaction and distillation are concurrently taking place at boiling point conditions. In other embodiments, the catalytic distillation reaction system may comprise a distillation column combined with at least one side reactor, where the side reactor may be operated as a liquid phase reactor or a boiling point reactor. While both catalytic distillation reaction systems described may be preferred over conventional liquid phase reaction followed by separations, a catalytic distillation column reactor may have the advantages of decreased piece count, reduced capital cost, increased catalyst productivity per pound of catalyst, efficient heat removal (heat of reaction may be absorbed into the heat of vaporization of the mixture), and a potential for shifting equilibrium.

The hydrocarbon feed to the reactor(s) may include purified isoolefin streams, such as a feed stream containing, isobutylene, isoamylenes, or mixtures thereof. In other embodiments, hydrocarbon feeds may include a C₄-C₅, a C₄, or a C₅ light naphtha cut. When present in mixtures, the tertiary olefins, such as isobutylene and isoamylenes, are more reactive than the normal olefin isomers and are preferentially reacted with alcohols to form ethers. The isoalkanes in the C₄ to C₅ light naphtha cuts may include isobutane, isopentane or mixtures thereof, which may act as a diluent in the reactors. The hydrocarbon feedstock may include up to 1 wt % 1,2-pentadiene or isoprene and 10-50 wt % isoamylene

In some embodiments, a C₄-containing hydrocarbon stream, such as a C₄ naphtha cut, a C₄-C₅ naphtha cut, or a C₄-C₆ naphtha cut may be fed to a reactor for the isomerization of 1-butene to 2-butene, thus allowing for the hydrogenation of butadiene to form additional 2-butene. The isomerization may be carried out in a fixed bed reactor as well as in a catalytic distillation reaction system. For example, in some embodiments, a feed containing 1-butene, 2-butene, butadiene, isobutylene, n-butane, and isobutane may be fed to a reaction system containing at least one bed of a multifunctional catalyst for the concurrent isomerization of 1-butene to 2-butene and hydrogenation of butadiene to 2-butenes, n-butane and other hydrogenation products.

The resulting product, including the residual 1-butene, 2-butene, isobutene, and any butanes, may be lean in 1-butene. For example, depending upon the severity of the reaction conditions used, the product may contain less than 1 weight percent total of 1-butene, less than 0.5 weight percent total in other embodiments; less than 0.1 weight percent total in other embodiments; and less than 500 ppm total in yet other embodiments.

Such a product may be suitable for etherification of the isobutylene and one or more alcohols to form one or more C₄ ethers such as MTBE and/or ETBE. Such above processes may also be suitable for similar reactions and conversions of a mixed C₅ stream to C₅ ethers such as TAME and/or TAEE.

The C₄ and/or C₅ isoolefins may be processed according to embodiments herein to etherify the isoolefins. Catalysts used in reactors and distillation column reactors according to embodiments herein may have functionality to selectively hydrogenate butadiene, isomerize olefins, as well as to etherify the isoolefins.

Typical conditions for the catalytic distillation MTBE reaction include catalyst bed temperatures above about 60° C., overhead pressures of above about 5.5 barg and equivalent liquid hourly space velocities of about 1.0 to 2.0 hr⁻¹. The temperature in the column is determined by the boiling point of the liquid mixture present at any given pressure. The temperature in the lower portions of the column will reflect the constitution of the material in that portion of the column, which will be higher than the overhead; that is, at constant pressure a change in the temperature indicates a change in the composition in the column. To change the temperature, the pressure in the column may be changed. Temperature control in the reaction zone is thus controlled by the pressure with the addition of heat (the reactions being exothermic) only causing more boil up. By increasing the pressure the temperature is increased, and vice versa. Even though a distillation column reactor is used, some of the isoolefin may be unconverted and may exit the column with the overheads.

The ether product, being the highest boiling material, is removed from the distillation column reactor as a bottoms, along with any dimers in the effluent from the upstream reactors. The overheads may contain unreacted light alcohols, such as methanol or ethanol used in the upstream reactors and/or a reactant in the distillation column reactor, and isoolefin along with light inerts, such as normal butenes and butanes or pentenes and pentanes.

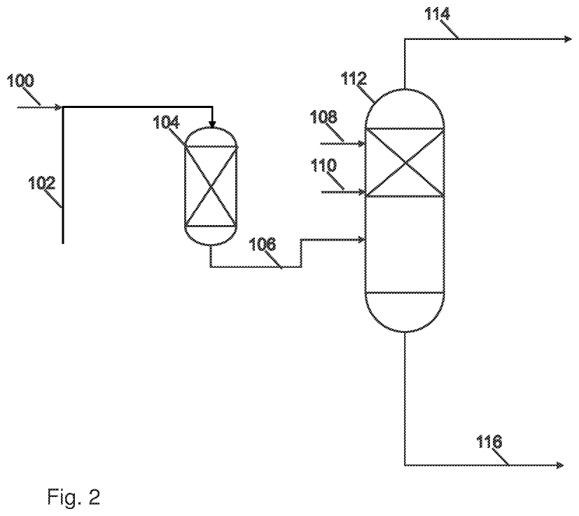

As an exemplary embodiment, the process for the production of methyl tertiary butyl ether (MTBE) will be discussed, and is illustrated in FIG. 2. The process involves isomerization of olefins, hydrogenation of the butadiene, and etherification of isoolefins.

A mixed C₄ stream 100 including alkanes, isoalkanes, 1-butene, 2-butene, butadiene, and isobutene, such as up to 1.2 wt % butadiene and 10-50 wt % isobutene, may be combined with an alcohol stream 102 including methanol to a fixed bed reactor 104 containing an etherification catalyst capable of reacting the isobutene and alcohol to form MTBE. In one or more embodiments, the mixed C₄ stream 100 may include up to 5 wt % n-butenes and 10-50 wt % isobutene.

The catalyst for the etherification may be any of known etherification catalysts such as an acidic cation exchange resin such as Amberlyst 15 as supplied by DuPont Chemical Company. A suitable catalytic structure may be used herein to place the cation exchange resin particles into a bed within the fixed bed reactor. Further, the temperatures and pressures may be similar to those known in the art for performing the specified reactions.

The effluent 106 from fixed bed reactor 104 may include alkanes, 1-butene, 2-butene, butadiene, and isobutene, as well as MTBE produced in the first fixed bed reactor. The effluent 106 may be fed to a catalyst distillation column reaction system 112, together with a second alcohol stream 108, and a hydrogen feed stream 110. The catalytic distillation reaction system 112 may have a single bed of a trifunctional catalyst capable of concurrent hydrogenation of butadiene, isomerization of 1-butene to 2-butene, and etherification of isobutene and the alcohol to form additional MTBE. The catalyst may be similar to the etherification catalyst, but including a hydrogenation catalyst and a basic metal oxide isomerization catalysts known in the art. For example, the trifunctional catalyst system may be an ion exchange resin doped with palladium (isomerization) and noble metal (hydrogenation) catalysts providing for the multi-functionality. In some embodiments, the catalyst distillation reaction system may be equipped with more than one bed of the trifunctional catalyst.

In the catalytic distillation reaction system 112, for the etherification reaction, the isobutene preferentially reacts with methanol in the reaction distillation zone to form additional methyl tertiary butyl ether which is higher boiling than either the C₄s or the methanol and so is distilled downward into the stripping section where any C₄s and methanol are boiled back up into the reaction distillation zone for further reaction.

MTBE is withdrawn from the catalytic distillation reaction system 112 as bottoms via flow line 116. The overheads contain mostly unreacted C₄s, excess hydrogen and other lighter hydrocarbons. The overheads are taken via flow line 114. The overhead C₄ stream may contain less than about 100 wppm of butadiene and in some cases only about 20 wppm. Further, the overhead may contain less than 100 wppm MTBE. The overhead products may be fed to any number of downstream processes, such as but not limited to, water washing to recover unreacted methanol/alcohol for recycle, a separation system to separate olefins from alkanes/paraffins, recycling the olefins to the upstream process, and sending the alkanes/paraffins to gasoline blending, alkylation units, or other hydrocarbon processes.

For the hydrogenation reaction, as with the etherification reaction, catalytic distillation is a benefit first, because the reaction is occurring concurrently with distillation, the initial reaction products and other stream components are removed from the reaction zone as quickly as possible reducing the likelihood of side reactions. Second, because all the components are boiling, the temperature of reaction is controlled at the boiling point of the mixture at the system pressure, the heat of reaction simply creates more boil up, but essentially no increase in temperature at a given pressure. As a result, a great deal of control over the rate of reaction and distribution of products can be achieved by regulating the system pressure. Also, adjusting the throughput (residence time=liquid hourly space velocity⁻¹) gives further control of product distribution and to a degree control of the side reactions such as oligomerization. A further benefit that this reaction may gain from catalytic distillation is the washing effect that the internal reflux provides to the catalyst thereby reducing polymer build up and coking. Internal reflux over the range of 0.4 to 5 L/D (wt. of liquid just below the catalyst bed/wt. distillate) gives excellent results.

For hydrogenation, the excess hydrogen, other lighter hydrocarbons, unreacted C₄ olefins, including butadiene, and an azeotrope of methanol (about 4%) are boiled upward into the reaction distillation zone wherein the butadiene reacts with the hydrogen to reduce butadiene content to about 20-100 wppm. In this manner, butadiene is hydrogenated to produce n-butenes and n-butanes.

The hydrogen stream at a hydrogen partial pressure of about 0.1 psia to 70 psia, may be fed to the reaction distillation column along with the other reactants, or at a height within the reactor above the effluent 106. Within the hydrogen partial pressures as defined no more hydrogen than necessary to hydrogenate the highly unsaturated compounds (dienes) is employed, since the excess hydrogen is usually vented.

Isomerization converts various alpha-olefins, such as 1-butene, to internal olefins, such as 2-butene. While described with respect to butenes, conversion of 1-pentene to 2-pentene, and the like, are also contemplated.

Reaction conditions disclosed herein may be carried out in a temperature range from 5° C. to about 500° C., such as at a temperature in the range from 250° C. to about 450° C. The reaction may also be controlled at a pressure in the range from atmospheric pressure to about 140 barg, such as atmospheric to 100 barg, or atmospheric to 50 barg. The reaction may also be controlled from about 3.5 barg or 6 barg to about 35 barg. The pressurization may also optionally be under an inert atmosphere. The temperatures and pressures may be suitable for etherification, isomerization, and hydrogenation concurrently.

The trifunctional catalyst may be located in a single bed within the catalyst distillation reaction system, or may be located in multiple beds. Compared to prior art processes where a multifunctional catalyst is paired with a single function catalyst, or multiple single function catalysts are used, the single bed of trifunctional catalyst is a much simpler operation. For example, the order/height of the catalyst beds is not important as the entire bed functions for hydrogenation, isomerization, and etherification.

Embodiments with a single bed of trifunctional catalyst may have advantages over the prior art processes in that the design and configuration may be simpler. Additionally, catalyst loading operations may be simpler as there are fewer beds and types of catalysts to be places. However, in some embodiments, it may be desirable to include a secondary, smaller catalyst bed above or below the main bed of trifunctional catalyst. The secondary catalyst may be a single-function etherification catalyst, or a single-function hydrogenation catalyst. The type of catalyst, placement above or below the trifunctional catalyst, and the amount of the secondary catalyst may be determined based on the feedstock composition, availability of hydrogen and/or alcohol feedstock, and desired conversion of olefins to ethers.

One of the advantages to the instant process is that C₄s from a light naphtha cut may be used without pretreatment. This C₄ stream may contain up to about 50 wt % isobutene, with 500 ppm butadiene to 2.5 wt % butadiene, or more. The remainder of the stream is essentially butanes and normal butenes. In such processes, the average overall isobutene conversion for the process may be greater than 90 wt %. Further, the purity of MTBE in the product stream may be 90 wt % or higher, such as 92 wt % or higher, or 94 wt % or higher, including commercial grade MTBE products.

While discussed above primarily as a process involving mixed C₄s for the etherification of isobutene with methanol to form MTBE, the process may also be extended to Os and methanol, C₄s and ethanol, Os and ethanol, higher hydrocarbons, higher primary alcohols, and combinations therefore.

For example, the same process steps may be used to convert isobutene and ethanol to produce ETBE, covert isoamylene and methanol to produce TAME, covert isoamylene and ethanol to produce TAEE, or a combination thereof. The same process steps may be used to covert a mixture of C₄s and C₅s, and a mixture of methanol and ethanol, to produce a mixture of two or more of MTBE, ETBE, TAME, and/or TAEE.

Advantageously, processes and systems according to one or more embodiments disclosed herein may have a reduced unit piece count as compared to prior art process. This reduced piece count may also come with reduced CAPEX and OPEX costs. Additionally, the system may have a lower overall H₂ partial pressure as there is no need to feed H₂ to any upstream hydrogenation reactor.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed as new and desired to be protected by Letters Patent of the United States is:

1. A process for the production of alkyl ethers, the process comprising:
feeding a hydrocarbon feedstock comprising n-alkanes, isoalkanes, alpha-olefins, internal-olefins, isoolefins, and diolefins and a first alcohol feedstock to a fixed bed reactor containing an etherification catalyst;
contacting the hydrocarbon feedstock and the first alcohol feedstock to react the isoolefins with the alcohol in the presence of the etherification catalyst to produce a first product stream comprising n-alkanes, isoalkanes, alpha-olefins, internal-olefins, unreacted isoolefins, diolefins, and unreacted alcohol and one or more ethers;
feeding the first product stream, a hydrogen feedstock, and a second alcohol feedstock to a catalytic distillation reaction system containing a trifunctional catalyst to concurrently:
isomerize at least a portion of the alpha-olefins, forming additional internal-olefins,
hydrogenate at least a portion of the diolefins, forming additional internal-olefins and alkanes;
etherify at least a portion of the isoolefins and alcohol, forming one or more ethers; and
produce a bottoms product comprising the one or more ethers; and
produce an overhead product comprising n-alkanes, isoalkanes, unreacted alpha-olefins, unreacted internal-olefins, unreacted isoolefins, excess hydrogen, and unreacted alcohol.

2. The process according to claim 1, further comprising separating the overheads stream, producing one or more of a saturated alkane stream, a residual olefin stream, and a residual alcohol stream.

3. The process according to claim 2, further comprising recycling the residual olefin stream to one or more of the fixed bed reactor and the catalytic distillation reaction system.

4. The process according to claim 2, further comprising recycling the residual alcohol stream to the one or more the fixed bed reactor and the catalytic distillation action system.

5. The process according to claim 1, further comprising operating the catalytic distillation reaction system at a pressure of between atmospheric pressure and 140 barg and a temperature of between 5° C. to about 500° C.

6. The process according to claim 1, wherein the hydrocarbon feedstock comprises up to 5 wt % n-butenes and 10-50 wt % isobutene.

7. The process according to claim 1, wherein the hydrocarbon feedstock comprises less than or equal to 1.2 wt % butadiene

8. The process according to claim 1, wherein the hydrocarbon feedstock comprises, up to 1 wt % 1,2-pentadiene or isoprene and 10-50 wt % isoamylene.

9. The process according to claim 1, wherein the first alcohol feedstock is methanol, ethanol, or a mixture thereof.

10. A system for the production of alkyl ethers, the system comprising:
a fixed bed reactor containing an etherification catalyst, the fixed bed reactor configured for receiving a hydrocarbon feedstock comprising n-alkanes, isoalkanes, alpha-olefins, internal-olefins, isoolefins, and diolefins and a first alcohol feedstock, and contacting the hydrocarbon feedstock and the first alcohol feedstock to produce a first product stream comprising n-alkanes, isoalkanes, alpha-olefins, internal-olefins, unreacted isoolefins, diolefins, and unreacted alcohol and one or more ethers;
a catalytic distillation reaction system containing a single bed of a trifunctional catalyst, the catalytic distillation reaction system further comprising:
a first product stream inlet located beneath the single bed of trifunctional catalyst;
a hydrogen feedstock inlet located proximate a top of the single bed of trifunctional catalyst;
a second alcohol feedstock inlet located proximate a bottom of the single bed of trifunctional catalyst;
a bottoms product outlet configured for producing a bottoms product comprising the one or more ethers; and
an overhead product outlet configured for producing an overhead product comprising n-alkanes, isoalkanes, unreacted alpha-olefins, unreacted internal-olefins, unreacted isoolefins, and unreacted alcohol;
the catalytic distillation reaction system configured to concurrently:
isomerize at least a portion of the alpha-olefins, forming additional internal-olefins,
hydrogenate at least a portion of the diolefins, forming additional internal-olefins; and
etherify at least a portion of the isoolefins and alcohol, forming one or more ethers.

11. The system according to claim 10, further comprising a separation system configured for separating the overheads product, producing one or more of a saturated alkane stream, a residual olefin stream, and a residual alcohol stream.

12. The system according to claim 10, further comprising an upstream separation system configured for producing a mixed C4 stream comprising up to 2.5 wt % butadiene and 10-50 wt % isobutene as the hydrocarbon feedstock.

13. The system according to claim 10, further comprising an upstream separation system configured for producing a mixed C5 hydrocarbon feedstock comprises up to 1 wt % pentadiene and 10-50 wt % isoamylene as the hydrocarbon feedstock.
